(19) United States
(12) Patent Application Publication
(10) Pub. No.: US 2020/0323430 A1
(43) Pub. Date: Oct. 15, 2020

(54) IMAGING DEVICE AND IMAGING METHOD

(71) Applicant: Shimadzu Corporation, Kyoto (JP)

(72) Inventor: Hiroyuki TSUMATORI, Kyoto (JP)

(21) Appl. No.: 16/843,583

(22) Filed: Apr. 8, 2020

(30) Foreign Application Priority Data
Apr. 9, 2019 (JP) 2019-074290

(51) Int. Cl. A61B 5/00; A61B 5/026

(52) U.S. Cl. CPC A61B 5/0033; A61B 5/0261; A61B 5/742; A61B 5/0082

(57) ABSTRACT

An imaging device and an imaging method are provided that are capable of, for example, accurately recognizing the state of blood circulation of an examinee when the examinee undergoes surgery. An imaging device includes: an excitation light source configured to irradiate an examinee with excitation light to excite a fluorescent dye administered to the examinee; a capturing unit configured to obtain a fluorescent image by capturing fluorescence generated from the fluorescent dye; a pixel value measurement unit configured to measure, over time, a pixel value of each pixel in a predetermined region of the fluorescent image; a gradation conversion unit configured to perform gradation conversion of the pixel values measured by the pixel value measurement unit to a predetermined number of gradations; a gradation image generation unit configured to generate a gradation image based on the pixel values subjected to the gradation conversion by the gradation conversion unit; and an image display unit configured to display the gradation image.

### BACKGROUND OF THE INVENTION

### 1. Field of the Invention

The present invention relates to an imaging device and an imaging method.

### 2. Description of the Related Art

A technique called near infrared fluorescence imaging is applied to angiography in surgery. In the near infrared fluorescence imaging, indocyanine green (ICG) as a fluorescent dye is injected by an injector or the like for administrated to an affected area. When such indocyanine green is irradiated with near infrared light with a wavelength approximately from 600 to 850 nm (nanometers) as excitation light, the indocyanine green generates near infrared fluorescence with a wavelength approximately from 750 to 900 nm. This fluorescence is captured by an image sensor capable of detecting near infrared light to display the captured image on a display unit, such as a liquid crystal display panel. Such near infrared fluorescence imaging allows observation of a blood vessel, a lymphatic vessel, and the like to a depth of approximately 20 mm from the body surface.

For example, Japanese Patent No. 5918532 discloses a method of generating a color image (color map) based on, by obtaining a graph illustrating temporal variation of fluorescence intensity, various indices such as the gradient, the time to peak, and the area of the graph. When the method described in Japanese Patent No. 5918532 is used, a color image is produced as, for example, an image of continuously changing colors in accordance with the state of blood circulation of an examinee. The doctor can determine, with reference to the color image, where the state of blood circulation is worsened, that is, where in the blood vessel to undergo a surgical treatment.

The color image obtained using the method described in Japanese Patent No. 5918532, however, causes continuous change of color in accordance with the blood circulation and thus causes difficulty in recognizing a boundary portion between good and bad states of blood circulation. Since the boundary portion between good and bad states of blood circulation is not determined, the area to cut off a blood vessel differs depending on the doctor.

### SUMMARY

The present invention has been made to solve the above problems, and it is an object of the present invention to provide an imaging device and an imaging method capable of accurately recognizing, for example, the state of blood circulation of an examinee when the examinee undergoes surgery.

A first aspect of the present invention is an imaging device including: an excitation light source configured to irradiate an examinee with excitation light to excite a fluorescent dye administered to the examinee; a capturing unit configured to obtain a fluorescent image by capturing fluorescence generated from the fluorescent dye; a pixel value measurement unit configured to measure, over time, a pixel value of each pixel in a predetermined region of the fluorescent image; a gradation conversion unit configured to perform gradation conversion of the pixel values measured by the pixel value measurement unit to a predetermined number of gradations; a gradation image generation unit configured to generate a gradation image based on the pixel values subjected to the gradation conversion by the gradation conversion unit; and an image display unit configured to display the gradation image.

A second aspect of the present invention is an imaging device including: an excitation light source configured to irradiate an examinee with excitation light to excite a fluorescent dye administered to the examinee; a capturing unit configured to obtain a fluorescent image by capturing fluorescence generated from the fluorescent dye; an image storage unit configured to store the fluorescent image over time; a pixel value measurement unit configured to measure, over time, a pixel value of each pixel in a predetermined region of the fluorescent image based on the fluorescent image stored in the image storage unit; a change curve creation unit configured to create a temporal change curve of the pixel value of each pixel in the predetermined region based on the pixel values measured by the pixel value measurement unit; an index calculation unit configured to analyze the change curve of each pixel in the predetermined region and calculate a predetermined index for each pixel in the predetermined region; a gradation conversion unit configured to perform gradation conversion of the predetermined index to a predetermined number of gradations; a gradation image generation unit configured to generate a gradation image based on the index subjected to the gradation conversion by the gradation conversion unit; and an image display unit configured to display the gradation image.

A third aspect of the present invention is an imaging method including the steps of: irradiating an examinee with excitation light to excite a fluorescent dye administered to the examinee; obtaining a fluorescent image by capturing fluorescence generated from the fluorescent dye; measuring, over time, a pixel value of each pixel in a predetermined region of the fluorescent image; performing gradation conversion of the measured pixel values to a predetermined number of gradations; generating a gradation image based on the pixel values subjected to the gradation conversion; and displaying the gradation image.

A fourth aspect of the present invention is an imaging method including the steps of: irradiating an examinee with excitation light to excite a fluorescent dye administered to the examinee; obtaining a fluorescent image by capturing fluorescence generated from the fluorescent dye; storing the fluorescent image over time; measuring, over time, a pixel value of each pixel in a predetermined region of the fluorescent image based on the fluorescent image subjected to the storing; creating a temporal change curve of the pixel value of each pixel in the predetermined region based on the measured pixel values; analyzing the change curve of each pixel in the predetermined region and calculating a predetermined index for each pixel in the predetermined region; performing gradation conversion of the predetermined index to a predetermined number of gradations; generating a gradation image based on the index subjected to the gradation conversion; and displaying the gradation image.

According to the present invention, the boundary between each gradation in the gradation image becomes clear. It is also possible to intuitively recognize the state of blood circulation by appropriately setting the color of each gradation. It is thus possible to accurately recognize, for example, the state of blood circulation of an examinee when the examinee undergoes surgery.

### BRIEF DESCRIPTION OF THE DRAWINGS

FIG. 1 is a perspective view illustrating an imaging device in a first embodiment of the present invention.

FIG. 2 is a side view of the imaging device illustrated in FIG. 1.

FIG. 3 is a plan view of the imaging device illustrated in FIG. 1.

FIG. 4 is a perspective view of an illumination and capturing unit provided in the imaging device illustrated in FIG. 1.

FIG. 5 is a schematic view of a capturing unit in the illumination and capturing unit illustrated in FIG. 4.

FIG. 6 is a block diagram illustrating a major control system of the imaging device illustrated in FIG. 1.

FIG. 7 is a drawing (flow chart) sequentially illustrating the steps executed by the imaging device illustrated in FIG. 1.

FIG. 8 is an example of an image obtained by executing the steps illustrated in FIG. 7.

FIG. 9 is another example of an image obtained by executing the steps illustrated in FIG. 7.

FIG. 10 is still another example of an image obtained by executing the steps illustrated in FIG. 7.

FIG. 11 is still another example of an image obtained by executing the steps illustrated in FIG. 7.

FIG. 12 is a block diagram illustrating a major control system of an imaging device (second embodiment) of the present invention.

FIG. 13 is a drawing sequentially illustrating the steps executed by the imaging device illustrated in FIG. 12.

FIG. 14 is a graph illustrating temporal variation of a pixel value obtained from TIC analysis by the imaging device illustrated in FIG. 12.

FIG. 15 is an example of an image obtained by an imaging device (third embodiment) of the present invention.

FIG. 16 is a graph illustrating temporal variation of a pixel value obtained from TIC analysis by the imaging device illustrated in FIG. 15.

### DETAILED DESCRIPTION OF THE PREFERRED EMBODIMENTS

The imaging device and the imaging method of the present invention are described below in detail based on preferred embodiments illustrated in the attached drawings.

### First Embodiment

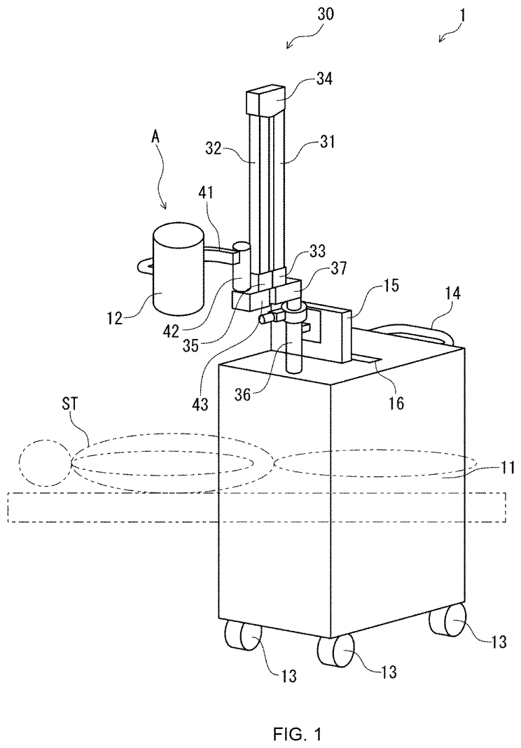

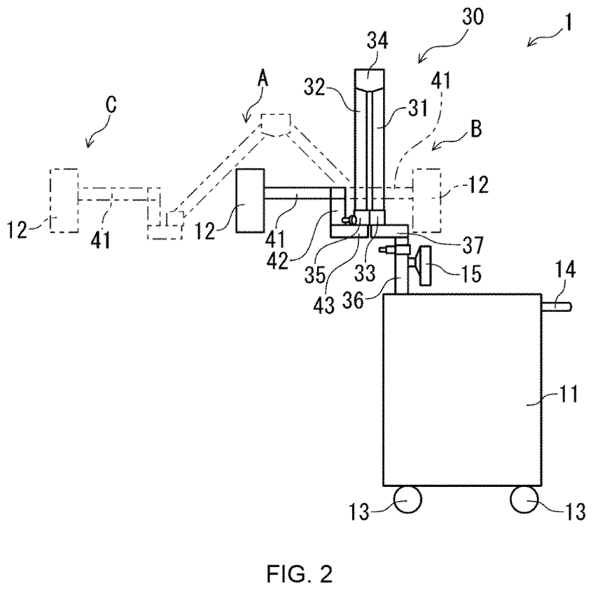

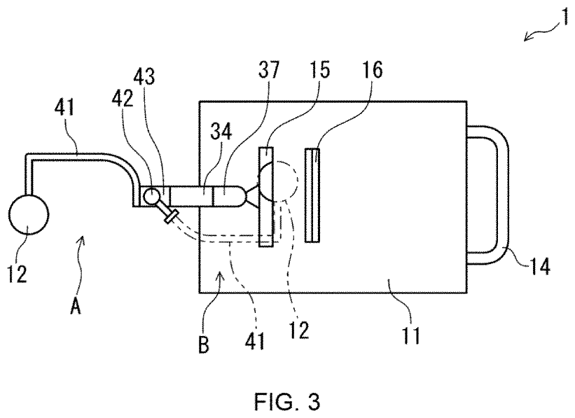

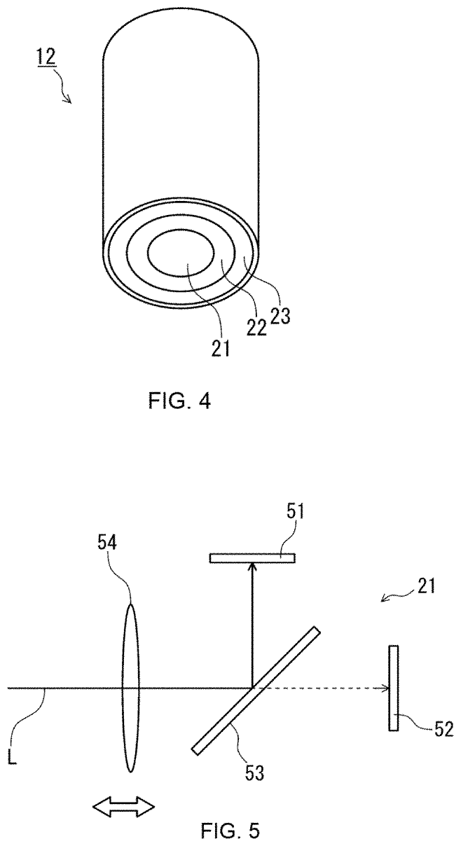

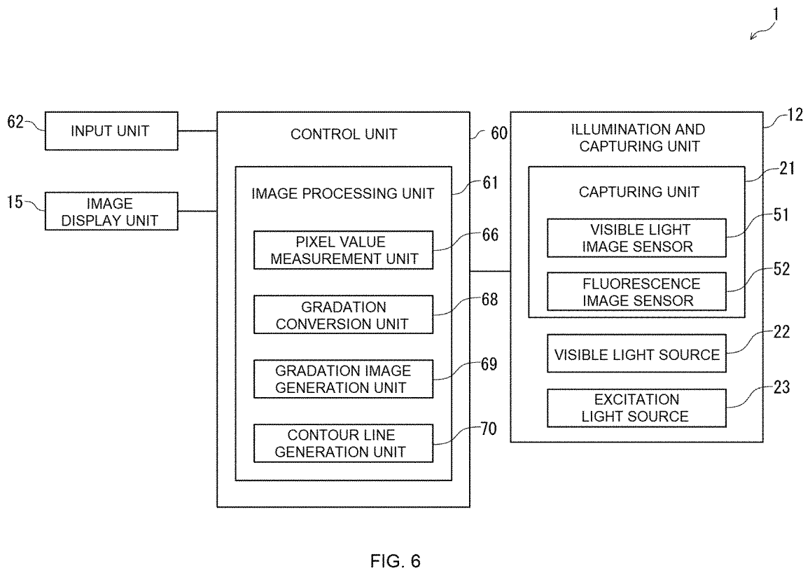

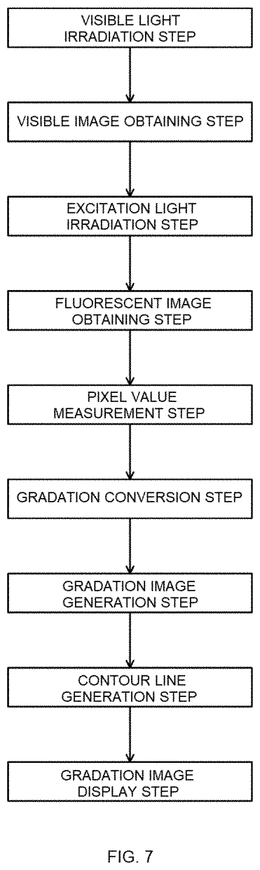

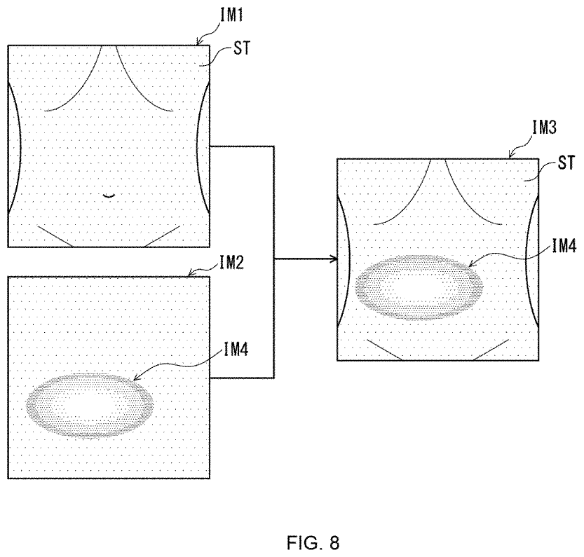

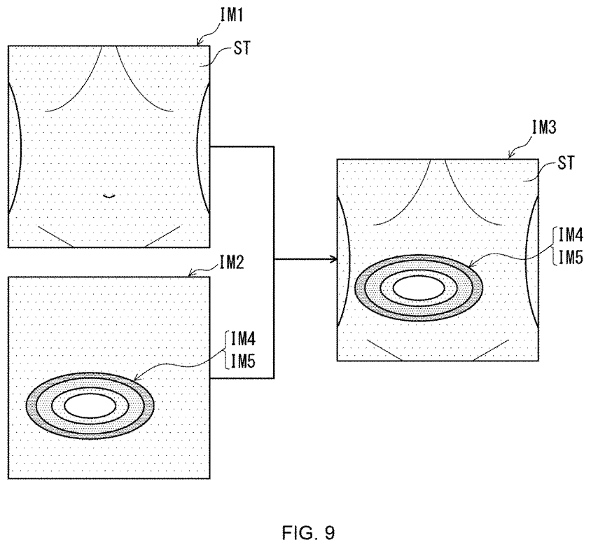

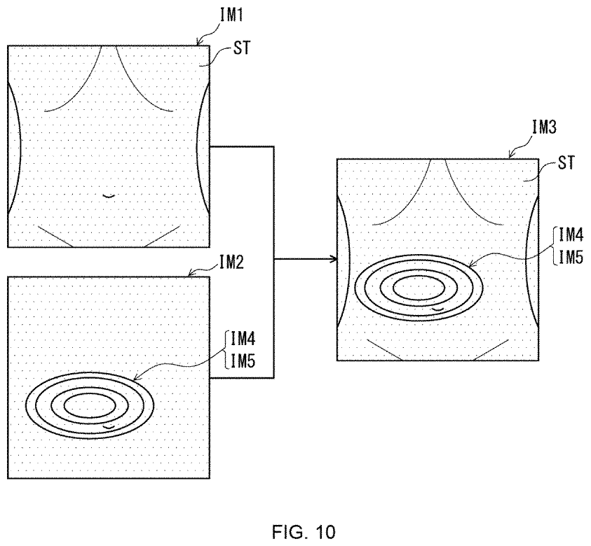

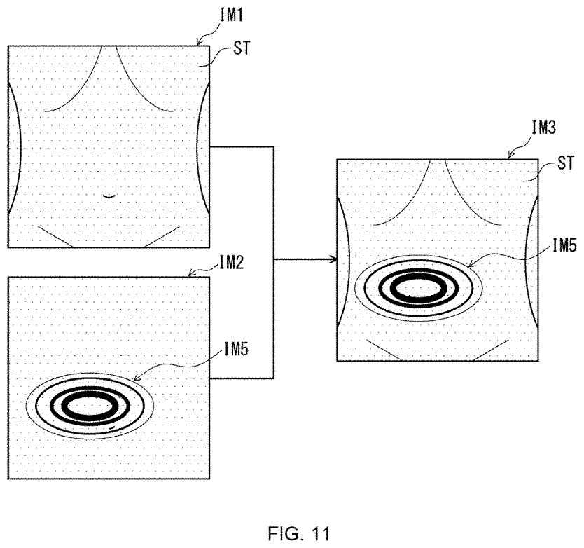

FIG. 1 is a perspective view illustrating an imaging device in the first embodiment of the present invention. FIG. 2 is a side view of the imaging device illustrated in FIG. 1. FIG. 3 is a plan view of the imaging device illustrated in FIG. 1. FIG. 4 is a perspective view of an illumination and capturing unit provided in the imaging device illustrated in FIG. 1. FIG. 5 is a schematic view of a capturing unit in the illumination and capturing unit illustrated in FIG. 4. FIG. 6 is a block diagram illustrating a major control system of the imaging device illustrated in FIG. 1. FIG. 7 is a drawing sequentially illustrating the steps executed by the imaging device illustrated in FIG. 1. FIG. 8 is an example of an image obtained by executing the steps illustrated in FIG. 7. FIG. 9 is another example of an image obtained by executing the steps illustrated in FIG. 7. FIG. 10 is still another example of an image obtained by executing the steps illustrated in FIG. 7. FIG. 11 is still another example of an image obtained by executing the steps illustrated in FIG. 7. In the following description, for the convenience of description, the upper side in FIGS. 1, 2, and 4 is referred to as “top (above)” and the lower side as “bottom (below)”.

An imaging device 1 illustrated in FIG. 1 is a device to irradiate indocyanine green (ICG) as a fluorescent dye injected into the body of an examinee ST with excitation light and capture fluorescence emitted from the indocyanine green. Use of the imaging device 1 allows accurate recognition of, for example, the state of blood circulation of the examinee ST when the examinee ST undergoes surgery.

The imaging device 1 includes a cart 11 provided with four wheels 13, an arm mechanism 30 arranged around a front side (on the left in FIGS. 2 and 3) in a travel direction of the cart 11 on a top surface of the cart 11, an illumination and capturing unit 12 arranged in the arm mechanism 30 via a sub-arm 41, and an image display unit 15 as a monitor. To a rear side in the travel direction of the cart 11, a handle 14 used to move the cart 11 is attached. On the top surface of the cart 11, a recess 16 is formed to mount a remote controller for remote operation of the imaging device 1.

As illustrated in FIG. 7, the imaging device 1 is capable of executing an imaging method including the steps, in this order of steps, of visible light irradiating, visible image obtaining, excitation light irradiating, fluorescent image obtaining, pixel value measuring, gradation converting, gradation image generating, contour line generating, and gradation image displaying.

The arm mechanism 30 described above is arranged on the front side in the travel direction of the cart 11. The arm mechanism 30 is provided with a first arm member 31 coupled by a hinge 33 to a support portion 37 arranged on a column 36 set upright on the front side in the travel direction of the cart 11. The first arm member 31 is swingable relative to the cart 11 via the column 36 and the support portion 37 by the action of the hinge 33. The image display unit 15 described above is attached to the column 36.

The first arm member 31 has an upper end coupled to a second arm member 32 by a hinge 34. The second arm member 32 is swingable relative to the first arm member 31 by the action of the hinge 34. This allows the first arm member 31 and the second arm member 32 to be in a capturing position, as illustrated by imaginary lines of the reference sign C in FIG. 2, in which the first arm member 31 and the second arm member 32 are opened at a predetermined angle about the hinge 34 as a coupling portion between the first arm member 31 and the second arm member 32 and a standby position, as illustrated by solid lines of the reference sign A in FIGS. 1 through 3, in which the first arm member 31 and the second arm member 32 are arranged close to each other.

The second arm member 32 has a lower end coupled to a support portion 43 by a hinge 35. The support portion 43 is swingable relative to the second arm member 32 by the action of the hinge 35. The support portion 43 supports a rotating shaft 42. The sub-arm 41 supporting the illumination and capturing unit 12 then pivots about the rotating shaft 42 arranged at the distal end of the second arm member 32. This causes the illumination and capturing unit 12 to move, due to the pivoting of the sub-arm 41, between a front side position in the travel direction of the cart 11 with respect to the arm mechanism 30 for taking the capturing position as illustrated by the solid lines of the reference sign A in FIGS. 1 through 3 or the standby position as illustrated by the imaginary lines of the reference sign C in FIG. 2 and a rear side position in the travel direction of the cart 11 with respect to the arm mechanism 30 in a position to move the cart 11 as illustrated by imaginary lines of the reference sign B in FIGS. 2 and 3.

As illustrated in FIG. 4, the illumination and capturing unit 12 has a capturing unit 21 as a camera provided with a plurality of image sensors capable of capturing visible light and near infrared light, a visible light source 22 arranged on the outer periphery of the capturing unit 21, and an excitation light source 23 arranged on the outer periphery of the visible light source 22.

The visible light source 22 irradiates visible light such as, for example, white light. It is thus possible to perform the visible light irradiation step to irradiate the examinee ST with visible light.

The excitation light source 23 irradiates excitation light to excite the fluorescent dye. It is thus possible to perform the excitation light irradiation step to irradiate the examinee ST subjected to administration of a fluorescent dye with excitation light to excite the fluorescent dye. In the case of the fluorescent dye being indocyanine green, near infrared light with a wavelength of, for example, 810 nm is preferably used as the excitation light to excite the indocyanine green. From the indocyanine green irradiated with the near infrared light at 810 nm, near infrared light with a peak of 845 nm approximately is emitted as fluorescence.

It should be noted that, although the visible light source 22, the excitation light source 23, and the capturing unit 21 are integrated into the illumination and capturing unit 12 in the present embodiment, the visible light source 22 and the excitation light source 23 may be arranged separately from the capturing unit 21.

As illustrated in FIG. 5, the capturing unit 21 has a movable lens 54 to reciprocate for focusing, a wavelength selection filter 53, a visible light image sensor 51, and a fluorescence image sensor 52. The visible light image sensor 51 and the fluorescence image sensor 52 are configured with a CMOS or a CCD. The visible light and the fluorescence incident on the capturing unit 21 coaxially along its optical axis L pass through the movable lens 54 constituting a focusing mechanism and then reach the wavelength selection filter 53.

Out of the visible light and the fluorescence that are coaxial, the fluorescence passes through the wavelength selection filter 53 and is incident on the fluorescence image sensor 52. It is thus possible to perform the fluorescent image obtaining step to capture fluorescence generated from the fluorescent dye and obtain a fluorescent image IM2 of the examinee ST.

In addition, out of the visible light and the fluorescence that are coaxial, the visible light is reflected on the wavelength selection filter 53 and is incident on the visible light image sensor 51. It is thus possible to perform the visible image obtaining step to capture a visible image IM1 in real time in an area corresponding to the fluorescent image IM2 and obtain the visible image IM1 of the examinee ST. On the image display unit 15, a synthetic image IM3 is then displayed in which the visible image IM1 and the fluorescent image IM2 are synthesized.

In the illumination and capturing unit 12, by the action of the focusing mechanism including the movable lens 54, the visible light is focused on the visible light image sensor 51 and the fluorescence is focused on the fluorescence image sensor 52.

It should be noted that, although the visible image obtaining step and the fluorescent image obtaining step are executed in the order of the visible image obtaining step and then the fluorescent image obtaining step in the procedure illustrated in FIG. 7, the order of executing these steps is not limited to above and, for example, they may be performed in the order of the fluorescent image obtaining step and then the visible image obtaining step, that is, in an inverse order or the visible image obtaining step and the fluorescent image obtaining step may be performed in parallel, that is, in synchronization.

As illustrated in FIG. 6, the imaging device 1 includes a control unit 60 having a CPU to execute logical operation, a ROM to store an operation program necessary for device control, a RAM to temporarily store data or the like during control, and the like to control the entire device. The control unit 60 has an image processing unit 61 to execute various types of image processing to the visible image IM1 and the fluorescent image IM2. The image processing unit 61 is provided with a pixel value measurement unit 66 configured to measure, over time, a pixel value of each pixel in a predetermined region of the fluorescent image IM2, a gradation conversion unit 68 configured to perform gradation conversion of the pixel values measured by the pixel value measurement unit 66 to a predetermined number of gradations, a gradation image generation unit 69 configured to generate a gradation image IM4 based on the pixel values subjected to the gradation conversion by the gradation conversion unit 68, and a contour line generation unit 70 configured to detect a boundary between each gradation in the gradation image IM4 and generate a contour line image IM5 representing each gradation.

In this context, the “predetermined region of the fluorescent image IM2” means a region generally called as a region of interest (ROI). In the imaging device 1, operation of an input unit 62 allows arbitrary setting and change of the position, the size (range), and the like of the ROI. The ROI may be the entire screen area of the fluorescent image IM2 or may be part of the fluorescent image IM2. In the former case, that is, the case where the ROI is the entire screen area of the fluorescent image IM2, it is possible to prevent, for example, oversight of the state of blood circulation depending on the case of the examinee ST. In addition, in the case where the ROI is the entire screen area of the fluorescent image IM2, it is preferred to perform a process such as, for example, reduction in a frame rate and reduction in screen size. Examples of the case of reduction in frame rate include a change from 60 fps to 15 fps and the like. Examples of the case of reduction in screen size include a change from “1280×1024” to “640×512” and the like. In the latter case, that is, the case where the ROI is part of the fluorescent image IM2, it is possible to improve, for example, the image processing speed of the image processing unit 61.

The fluorescent dye administered to the examinee ST may be directly carried away without remaining in the ROI, or on the contrary, may mostly remain in part of the ROI. The pixel value of each pixel in the predetermined region of the fluorescent image IM2, that is, in the ROI is a value correlated to the concentration of the fluorescent dye in the ROI. The pixel value measurement unit 66 is capable of performing the pixel value measurement step to measure, over time, the pixel value of each pixel in the ROI.

The gradation conversion unit 68 is capable of performing the gradation conversion step to perform gradation conversion of the pixel values measured by the pixel value measurement unit 66 to a predetermined number of gradations. When the number of gradations is four, for example, the gradation conversion unit 68 sets four thresholds different in range (magnitude) to obtain the threshold in which range a pixel value measured by the pixel value measurement unit 66 belongs to. Based on this result, it is possible to determine which gradation out of the four gradations the pixel value belongs to. In the imaging device 1, the number of gradations and the ranges of thresholds may be arbitrarily set and changed by operating the input unit 62.

The gradation image generation unit 69 is capable of performing the gradation image generation step to generate the gradation image IM4 based on the pixel values subjected to the gradation conversion by the gradation conversion unit 68. In the case where the number of gradations in the gradation conversion unit 68 is four as described above, the gradation image generation unit 69 processes the image by, for example, classifying the pixel values belonging to each gradation into one respective group, to display the group as one continuous region. It is thus possible to generate the gradation image IM4 with the gradations divided into four, for example, as illustrated in FIGS. 8 and 9.

The contour line generation unit 70 is capable of performing the contour line generation step to generate the contour line image IM5 representing each gradation by detecting a boundary between each adjacent gradation in the gradation image IM4, that is, an edge as a contour of each gradation. The contour line generation unit 70 processes the image to display the detected boundary as a contour line surrounding each gradation. It is thus possible to generate the contour line image IM5 having four contour lines as illustrated in, for example, FIGS. 9 through 11.

The control unit 60 is electrically connected to the input unit 62 to input various types of information by an operator. The control unit 60 is also electrically connected to the image display unit 15 described above. The image display unit 15 is thus capable of performing the gradation image display step to display the synthetic image IM3 containing the gradation image IM4 and the like. The input unit 62 may be arranged in a remote controller for remote operation of the imaging device 1, or when the image display unit 15 is configured with a touch screen, may be arranged on the screen of the image display unit 15, or may be arranged in the cart 11.

The control unit 60 is also electrically connected to the illumination and capturing unit 12 provided with the capturing unit 21, the visible light source 22, and the excitation light source 23. The illumination and capturing unit 12 is thus capable of performing the visible light irradiation step, the visible image obtaining step, the excitation light irradiation step, and the fluorescent image obtaining step described above.

The case of recognizing the state of blood circulation of the examinee ST using the imaging device 1 for surgery on the examinee ST is then described with reference to the flow chart illustrated in FIG. 7 and the images illustrated in FIGS. 8 through 11. It should be noted that, prior to the surgery, the fluorescent dye is already administered to the examinee ST. In the imaging device 1, the position and size of the ROI are also set in advance.

First, the visible light source 22 is activated to establish a visible light irradiation state in which the examinee ST is irradiated with visible light (visible light irradiation step).

The capturing unit 21 is then activated while the visible light irradiation state is maintained to obtain the visible image IM1 (visible image obtaining step). The visible image IM1 as illustrated in FIGS. 8 through 11 is thus obtained. After the visible image IM1 is obtained, it is preferred to stop irradiation of visible light by the visible light source 22.

The excitation light source 23 is then activated to establish an excitation light irradiation state in which the examinee ST is irradiated with excitation light (excitation light irradiation step). It is thus possible to excite the fluorescent dye in the examinee ST and therefore to generate fluorescence from the fluorescent dye.

The capturing unit 21 is then activated while the excitation light irradiation state is maintained to obtain the fluorescent image IM2 (fluorescent image obtaining step). After the fluorescent image IM2 is obtained, it is preferred to stop irradiation of excitation light by the excitation light source 23.

Then, while the fluorescent image IM2 is obtained over time, the pixel value measurement unit 66 measures, over time, the pixel value of each pixel in the ROI in the fluorescent image IM2 (pixel value measurement step).

The gradation conversion unit 68 then performs gradation conversion of the pixel values measured by the pixel value measurement unit 66 to, for example, four gradations (gradation conversion step).

Then, based on the pixel values subjected to the gradation conversion by the gradation conversion unit 68, the gradation image generation unit 69 generates the gradation image IM4 (gradation image generation step). The fluorescent image IM2 containing the gradation image IM4 as illustrated in FIG. 8 is thus obtained.

In addition, the gradation image generation unit 69 is capable of generating the gradation image IM4 with a different color for each gradation. The color of each gradation is preferably selected based on, for example, HSV or HSL as a type of color space. Still in addition, the gradation image generation unit 69 is allowed to use, for example, complementary colors for colors of adjacent gradations. Such a gradation image IM4 causes the boundary between each gradation in the gradation image IM4 to become clear. It is also possible to intuitively recognize the state of blood circulation by appropriately setting the color of each gradation.

The contour line generation unit 70 then detects the boundary between each adjacent gradation in the gradation image IM4 to generate the contour line image IM5 representing each gradation (contour line generation step). The fluorescent image IM2 is thus obtained that contains the contour line image IM5 as illustrated in FIGS. 9 through 11. The contour line image IM5 is capable of emphasizing, that is, gaining more visibility for the boundary between adjacent gradations.

The contour line generation unit 70 is capable of generating the contour line image IM5 with a different color for each gradation, that is, different in color for each contour line. The color of each contour line is preferably selected based on, for example, HSV or HSL as a type of color space. In addition, the color of each contour line is preferably different from the color of each gradation. Such a contour line image IM5 is capable of more emphasis on the boundary between adjacent gradations.

The contour line generation unit 70 is also capable of generating the contour line image IM5 with a different thickness for each gradation, that is, different in thickness for each contour line. In this case, the fluorescent image IM2 is obtained that contains the contour line image IM5 as illustrated in FIG. 11. Such a contour line image IM5 allows the thickness of the contour line to reflect the brightness of each gradation. For example, the contour line may be relatively thick in a region of relatively bright gradation and the contour line may be relatively thin in a region of relatively dark gradation. This also allows more emphasis on the boundary between adjacent gradations.

The image display unit 15 then displays the synthetic image IM3 (gradation image display step). It is thus possible to display the synthetic image IM3 as illustrated in FIGS. 8 through 11 on the image display unit 15.

The synthetic image IM3 illustrated in FIG. 8 is an image where the gradation image IM4 is superimposed on the visible image IM1. This allows the doctor to readily visually recognize the boundary between each gradation in the gradation image IM4. Based on the boundary, the doctor can accurately recognize the boundary portion between good and bad states of blood circulation. This also allows the doctor, regardless of the level of the skills, to accurately determine the area to cut off a blood vessel, that is, a cut off line.

The synthetic image IM3 illustrated in FIG. 9 is an image where the gradation image IM4 and the contour line image IM5 are superimposed on the visible image IM1. This allows emphasizing the boundary between adjacent gradations in the gradation image IM4. Then, the doctor can more readily visually recognize the boundary between each gradation in the gradation image IM4.

The synthetic image IM3 illustrated in FIG. 10 is an image where the contour line image IM5 is superimposed on the visible image IM1. Such a synthetic image IM3 is suitable for the case of not using the gradation image IM4. This allows improvement in the image processing speed of the image processing unit 61.

The synthetic image IM3 illustrated in FIG. 11 is an image where the contour line image IM5 is superimposed on the visible image IM1 and is same as the synthetic image IM3 illustrated in FIG. 10 except for having a different thickness for each contour line. This allows emphasizing the boundary between adjacent gradations instead of the gradation image IM4, and thus the doctor can more readily visually recognize the boundary between each gradation.

As just described, use of the imaging device 1 for surgery on the examinee ST allows accurate recognition of the state of blood circulation of the examinee ST. It is thus possible to safely perform an operation on the examinee ST.

In addition, the imaging device 1 is capable of displaying at least any one of the gradation image IM4 or the contour line image IM5 or the gradation image IM4 and the contour line image IM5 by switching them on the image display unit 15. This allows the doctor to select the synthetic image IM3 containing the gradation image IM4, the synthetic image IM3 containing the contour line image IM5, or the synthetic image IM3 containing the gradation image IM4 and the contour line image IM5 in accordance with the preference to use for the operation.

### Second Embodiment

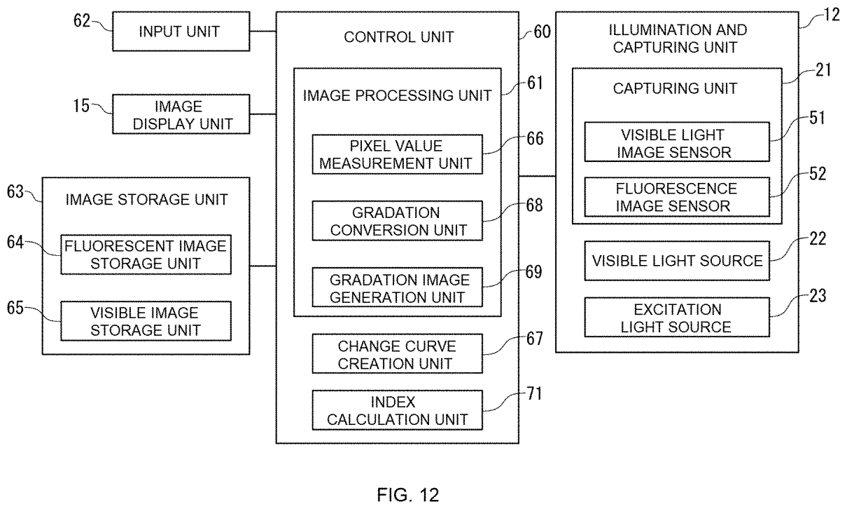

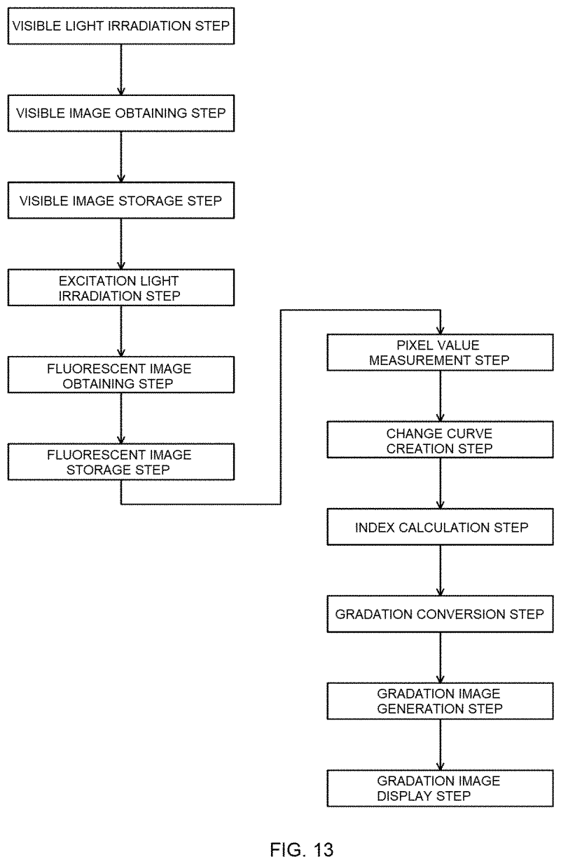

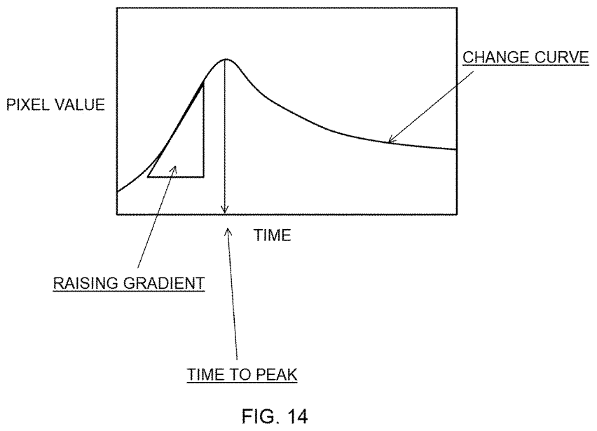

FIG. 12 is a block diagram illustrating a major control system of an imaging device (second embodiment) of the present invention. FIG. 13 is a drawing sequentially illustrating the steps executed by the imaging device illustrated in FIG. 12. FIG. 14 is a graph illustrating temporal variation of a pixel value obtained from TIC analysis by the imaging device illustrated in FIG. 12.

The imaging device and the imaging method in the second embodiment of the present invention are described below with reference to these drawings while the description is mainly given to the difference from the embodiment described earlier to omit the description on similar points.

The present embodiment is same as the first embodiment except for the imaging device different in configuration.

As illustrated in FIG. 12, in the present embodiment, an imaging device 1 includes an image storage unit 63 configured to store, over time, an image captured by the capturing unit 21. The image storage unit 63 is electrically connected to a control unit 60. The image storage unit 63 is configured with a fluorescent image storage unit 64 to store the fluorescent image IM2 over time and a visible image storage unit 65 to store the visible image IM1 over time. It should be noted that the image storage unit 63 may have, instead of the fluorescent image storage unit 64 and the visible image storage unit 65, a synthesis image storage unit to store the synthetic image IM3 over time.

The control unit 60 further has, in addition to the image processing unit 61, a change curve creation unit 67 configured to create a temporal change curve of a pixel value of each pixel in the ROI based on the pixel values measured by the pixel value measurement unit 66 and an index calculation unit 71 configured to analyze the change curve of each pixel in the ROI and calculate a predetermined index for each pixel in the ROI.

As illustrated in FIG. 13, the imaging device 1 is capable of executing an imaging method including the steps, in this order of steps, of visible light irradiating, visible image obtaining, visible image storing, excitation light irradiating, fluorescent image obtaining, fluorescent image storing, pixel value measuring, change curve creating, index calculating, gradation converting, gradation image generating, and gradation image displaying.

The visible image storage step is a step of storing the visible image IM1 over time and is performed by the visible image storage unit 65.

The fluorescent image storage step is a step of storing the fluorescent image IM2 over time and is performed by the fluorescent image storage unit 64. The pixel value measurement unit 66 is then capable of measuring, over time, the pixel value of each pixel in the ROI of the fluorescent image IM2 as described above based on the fluorescent image IM2 stored in the image storage unit 63 (pixel value measurement step).

The change curve creation step is a step of creating a temporal change curve of the pixel value of each pixel in the ROI as illustrated in FIG. 14 based on the pixel values measured by the pixel value measurement unit 66 and is performed by the change curve creation unit 67. The change curve creation unit 67 displays, for example, the pixel values in the ROI as a graph.

The index calculation step is a step of analyzing the change curve of each pixel in the ROI and calculating a predetermined index for each pixel in the ROI and is performed by the index calculation unit 71. The predetermined index is an index to recognize the state of blood flow, and a preferred example of the index is any one of, but not particularly limited to, gradient of the change curve, a peak value, the time to peak, an integral, a curve shape, fluorescence starting time (the time of first detecting fluorescence in each pixel), or a final pixel value (stabilized pixel value after reaching the peak and then attenuated) of time intensity curve (TIC) analysis.

The index calculation unit 71 is capable of calculating any one of the above indices by TIC analysis. For example, the “raising gradient” in FIG. 14 is a parameter correlated to the blood flow, and the “time to peak” is a parameter (time) between administration of the fluorescent dye and delivery of the fluorescent dye to a target tissue. Then, it is finally possible to recognize the state of blood circulation, that is, how well the blood flows from any of the parameters.

The gradation conversion unit 68 is capable of performing gradation conversion of the predetermined index to a predetermined number of gradations (gradation conversion step). The gradation image generation unit 69 is capable of generating the gradation image IM4 based on the index subjected to the gradation conversion by the gradation conversion unit 68 (gradation image generation step). It is thus possible to, similar to the first embodiment, accurately recognize the state of blood circulation of the examinee ST when the examinee ST undergoes surgery.

### Third Embodiment

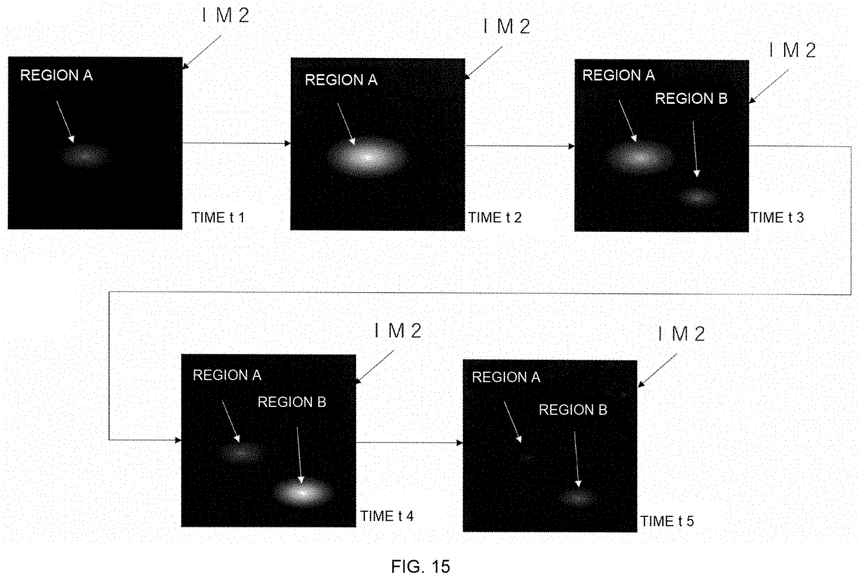

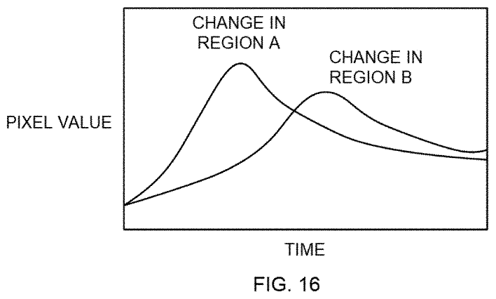

FIG. 15 is an example of an image obtained by an imaging device (third embodiment) of the present invention. FIG. 16 is a graph illustrating temporal variation of a pixel value obtained from TIC analysis by the imaging device illustrated in FIG. 15.

The imaging device and the imaging method in the third embodiment of the present invention are described below with reference to these drawings, and the description is mainly given to the difference from the embodiments described earlier to omit the description on similar points.

The present embodiment is same as the second embodiment except for the difference in the number of ROI set in the fluorescent image IM2.

As illustrated in FIG. 15, in the present embodiment, two regions (region A and region B) are set as ROIs in the fluorescent image IM2. As illustrated in FIG. 16, in the fluorescent image IM2, fluorescence is generated in the region A, and after a delay, that is, after a time lag, fluorescence is generated in the region B. In this case, for example, measurement of the time lag in fluorescence generation between the region A and the region B allows recognition of the state of blood circulation.

It should be noted that the number of ROI set in the fluorescent image IM2 is not limited to two as in the present embodiment and may be, for example, three or more.

Although the imaging device and the imaging method of the present invention has been described above with reference to the illustrated embodiments, the present invention is not limited to above and each component constituting the imaging device and the imaging method may be substituted by an arbitrary configuration capable of exhibiting the same function. In addition, an arbitrary configuration may be added.

The imaging device of the present invention may be a combination of two or more arbitrary configurations (features) in the respective embodiments above. For example, the imaging device in the second embodiment may be configured to include a contour line generation unit same as that of the imaging device in the first embodiment.

Although the respective embodiments above are described for the case of emitting fluorescence in the near infrared region with a peak of roughly 810 nm from indocyanine green by using the indocyanine green as a material containing the fluorescent dye and irradiating the indocyanine green with near infrared light approximately from 600 nm to 850 nm as excitation light, light other than near infrared rays may be used.

Depending on the case of the examinee, 5-aminolevulinic acid (5-ALA), for example, may be used as the fluorescent dye instead of the indocyanine green.

### Aspects

Those skilled in the art understand that the plurality of embodiments described above as exemplifications are specific examples of the following aspects.

First Aspect The imaging device according to an aspect includes:

an excitation light source configured to irradiate an examinee with excitation light to excite a fluorescent dye administered to the examinee;

a capturing unit configured to obtain a fluorescent image by capturing fluorescence generated from the fluorescent dye;

a pixel value measurement unit configured to measure, over time, a pixel value of each pixel in a predetermined region of the fluorescent image;

a gradation conversion unit configured to perform gradation conversion of the pixel values measured by the pixel value measurement unit to a predetermined number of gradations;

a gradation image generation unit configured to generate a gradation image based on the pixel values subjected to the gradation conversion by the gradation conversion unit; and

an image display unit configured to display the gradation image.

In accordance with the imaging device according to the first aspect, the boundary between each gradation in the gradation image becomes clear. It is also possible to intuitively recognize the state of blood circulation by appropriately setting the color of each gradation. It is thus possible to accurately recognize, for example, the state of blood circulation of an examinee when the examinee undergoes surgery.

Second Aspect In the imaging device according to the first aspect,

the gradation image generation unit generates the gradation image with a different color for each gradation.

In accordance with the imaging device according to the second aspect, the boundary between each gradation in the gradation image becomes clear.

Third Aspect In the imaging device according to the first or second aspect,

the imaging device further includes a contour line generation unit configured to detect a boundary between each gradation in the gradation image and generate a contour line image representing each gradation, wherein

the image display unit displays at least any one of the gradation image or the contour line image.

In accordance with the imaging device according to the third aspect, it is possible to readily visually recognize the state of blood circulation.

Fourth Aspect In the imaging device according to the third aspect,

the contour line generation unit generates the contour line image with a different thickness for each gradation.

In accordance with the imaging device according to the fourth aspect, it is possible to reflect the brightness of each gradation on the thickness of the contour line.

Fifth Aspect In the imaging device according to the third or fourth aspect,

the contour line generation unit generates the contour line image with a different color for each gradation.

In accordance with the imaging device according to the fifth aspect, the boundary between each gradation in the gradation image becomes clear.

Sixth Aspect In the imaging device according to any one of the first through fifth aspects,

the predetermined region is an entire screen area of the fluorescent image.

In accordance with the imaging device according to the sixth aspect, it is possible to prevent, for example, oversight of the state of blood circulation depending on the case of the examinee.

Seventh Aspect In the imaging device according to any one of the first through fifth aspects,

the predetermined region is a plurality of regions in the fluorescent image.

In accordance with the imaging device according to the seventh aspect, it is possible to improve, for example, the image processing speed.

Eighth Aspect In the imaging device according to any one of the first through seventh aspects,

the capturing unit captures a visible image in real time in an area corresponding to the fluorescent image, and

the image display unit superimposes the gradation image on the visible image for display.

In accordance with the imaging device according to the eighth aspect, it is possible to readily visually recognize the boundary between each gradation in the gradation image.

Ninth Aspect An imaging device according to an aspect includes:

an excitation light source configured to irradiate an examinee with excitation light to excite a fluorescent dye administered to the examinee;

a capturing unit configured to obtain a fluorescent image by capturing fluorescence generated from the fluorescent dye;

an image storage unit configured to store the fluorescent image over time;

a pixel value measurement unit configured to measure, over time, a pixel value of each pixel in a predetermined region of the fluorescent image based on the fluorescent image stored in the image storage unit;

a change curve creation unit configured to create a temporal change curve of the pixel value of each pixel in the predetermined region based on the pixel values measured by the pixel value measurement unit;

an index calculation unit configured to analyze the change curve of each pixel in the predetermined region and calculate a predetermined index for each pixel in the predetermined region;

a gradation conversion unit configured to perform gradation conversion of the predetermined index to a predetermined number of gradations;

a gradation image generation unit configured to generate a gradation image based on the index subjected to the gradation conversion by the gradation conversion unit; and

an image display unit configured to display the gradation image.

In accordance with the imaging device according to the ninth aspect, the boundary between each gradation in the gradation image becomes clear. It is also possible to intuitively recognize the state of blood circulation by appropriately setting the color of each gradation. It is thus possible to accurately recognize, for example, the state of blood circulation of an examinee when the examinee undergoes surgery.

Tenth Aspect In the imaging device according to the ninth aspect,

the predetermined index is any one of gradient, a peak value, time to peak, an integral, or a curve shape of the change curve.

In accordance with the imaging device according to the tenth aspect, it is possible to stably recognize the state of blood flow.

Eleventh Aspect In the imaging device according to the ninth or tenth aspect,

the gradation image generation unit generates the gradation image with a different color for each gradation.

In accordance with the imaging device according to the eleventh aspect, the boundary between each gradation in the gradation image becomes clear.

Twelfth Aspect In the imaging device according to any one of the ninth through eleventh aspects,

the imaging device further includes a contour line generation unit configured to detect a boundary between each gradation in the gradation image and generate a contour line image representing each gradation, wherein

the image display unit is capable of switching between the gradation image and the contour line image for display.

In accordance with the imaging device according to the twelfth aspect, it is possible to switch the image in accordance with the state of blood circulation and thus to readily visually recognize the state of blood circulation.

Thirteenth Aspect In the imaging device according to the twelfth aspect,

the contour line generation unit generates the contour line image with a different thickness for each gradation.

In accordance with the imaging device according to the thirteenth aspect, it is possible to reflect the brightness of each gradation on the thickness of the contour line.

Fourteenth Aspect In the imaging device according to the twelfth or thirteenth aspect,

the contour line generation unit generates the contour line image with a different color for each gradation.

In accordance with the imaging device according to the fourteenth aspect, the boundary between each gradation in the gradation image becomes clear.

Fifteenth Aspect In the imaging device according to any one of the ninth through fourteenth aspects,

the predetermined region is an entire screen area of the fluorescent image.

In accordance with the imaging device according to the fifteenth aspect, it is possible to prevent, for example, oversight of the state of blood circulation depending on the case of the examinee.

Sixteenth Aspect In the imaging device according to any one of the ninth through fourteenth aspects,

the predetermined region is a plurality of regions in the fluorescent image.

In accordance with the imaging device according to the sixteenth aspect, it is possible to improve, for example, the image processing speed.

Seventeenth Aspect In the imaging device according to any one of the ninth through sixteenth aspects,

the capturing unit captures a visible image in real time in an area corresponding to the fluorescent image, and

the image display unit superimposes the gradation image on the visible image for display.

In accordance with the imaging device according to the seventeenth aspect, it is possible to readily visually recognize the boundary between each gradation in the gradation image.

Eighteenth Aspect An imaging method according to an aspect includes the steps of:

irradiating an examinee with excitation light to excite a fluorescent dye administered to the examinee;

obtaining a fluorescent image by capturing fluorescence generated from the fluorescent dye;

measuring, over time, a pixel value of each pixel in a predetermined region of the fluorescent image;

performing gradation conversion of the measured pixel values to a predetermined number of gradations;

generating a gradation image based on the pixel values subjected to the gradation conversion; and

displaying the gradation image.

In accordance with the imaging method according to the eighteenth aspect, the boundary between each gradation in the gradation image becomes clear. It is also possible to intuitively recognize the state of blood circulation by appropriately setting the color of each gradation. It is thus possible to accurately recognize, for example, the state of blood circulation of an examinee when the examinee undergoes surgery.

Nineteenth Aspect In the imaging method according to the eighteenth aspect,

the generating a gradation image generates the gradation image with a different color for each gradation.

In accordance with the imaging method according to the nineteenth aspect, the boundary between each gradation in the gradation image becomes clear.

Twentieth Aspect In the imaging method according to the eighteenth or nineteenth aspect,

the imaging method further includes detecting a boundary between each gradation in the gradation image and generating a contour line image representing each gradation, wherein

the displaying a gradation image displays at least any one of the gradation image or the contour line image.

In accordance with the imaging method according to the twentieth aspect, it is possible to readily visually recognize the state of blood circulation.

Twenty-First Aspect In the imaging method according to the twelfth aspect,

the generating a contour line image generates the contour line image with a different thickness for each gradation.

In accordance with the imaging method according to the twenty-first aspect, it is possible to reflect the brightness of each gradation on the thickness of the contour line.

Twenty-Second Aspect In the imaging method according to the twelfth ore twenty-first aspect,

the generating a contour line image generates the contour line image with a different color for each gradation.

In accordance with the imaging method according to the twenty-second aspect, the boundary between each gradation in the gradation image becomes clear.

Twenty-Third Aspect In the imaging method according to any one of the eighteenth through twenty-second aspects,

the predetermined region is an entire screen area of the fluorescent image.

In accordance with the imaging method according to the twenty-third aspect, it is possible to prevent, for example, oversight of the state of blood circulation depending on the case of the examinee.

Twenty-Fourth Aspect In the imaging method according to any one of the eighteenth through twenty-second aspects,

the predetermined region is a plurality of regions in the fluorescent image.

In accordance with the imaging method according to the twenty-fourth aspect, it is possible to improve, for example, the image processing speed.

Twenty-Fifth Aspect In the imaging method according to any one of the eighteenth through twenty-fourth aspects,

the imaging method further includes capturing a visible image in real time in an area corresponding to the fluorescent image, wherein

the displaying a gradation image superimposes the gradation image on the visible image for display.

In accordance with the imaging method according to the twenty-fifth aspect, it is possible to readily visually recognize the boundary between each gradation in the gradation image.

Twenty-Sixth Aspect An imaging method according to an aspect includes the steps of:

irradiating an examinee with excitation light to excite a fluorescent dye administered to the examinee;

obtaining a fluorescent image by capturing fluorescence generated from the fluorescent dye;

storing the fluorescent image over time;

measuring, over time, a pixel value of each pixel in a predetermined region of the fluorescent image based on the fluorescent image subjected to the storing;

creating a temporal change curve of the pixel value of each pixel in the predetermined region based on the measured pixel values;

analyzing the change curve of each pixel in the predetermined region and calculating a predetermined index for each pixel in the predetermined region;

performing gradation conversion of the predetermined index to a predetermined number of gradations;

generating a gradation image based on the index subjected to the gradation conversion; and

displaying the gradation image.

In accordance with the imaging method according to the twenty-sixth aspect, the boundary between each gradation in the gradation image becomes clear. It is also possible to intuitively recognize the state of blood circulation by appropriately setting the color of each gradation. It is thus possible to accurately recognize, for example, the state of blood circulation of an examinee when the examinee undergoes surgery.

Twenty-Seventh Aspect In the imaging method according to the twelfth aspect,

the predetermined index is any one of gradient, a peak value, time to peak, an integral, or a curve shape of the change curve.

In accordance with the imaging method according to the twenty-seventh aspect, it is possible to stably recognize the state of blood flow.

Twenty-Eighth Aspect In the imaging method according to the twenty-sixth or twenty-seventh aspect,

the generating a gradation image generates the gradation image with a different color for each gradation.

In accordance with the imaging method according to the twenty-eighth aspect, the boundary between each gradation in the gradation image becomes clear.

Twenty-Ninth Aspect In the imaging method according to any one of the twenty-sixth through twenty-eighth aspects,

the imaging method further includes detecting a boundary between each gradation in the gradation image and generating a contour line image representing each gradation, wherein

the displaying a gradation image is capable of switching between the gradation image and the contour line image for display.

In accordance with the imaging method according to the twenty-ninth aspect, it is possible to switch the image in accordance with the state of blood circulation and thus to readily visually recognize the state of blood circulation.

Thirtieth Aspect In the imaging method according to the twenty-ninth aspect,

the generating a contour line image generates the contour line image with a different thickness for each gradation.

In accordance with the imaging method according to the thirtieth aspect, it is possible to reflect the brightness of each gradation on the thickness of the contour line.

Thirty-First Aspect In the imaging method according to the twenty-ninth or thirtieth aspect,

the generating a contour line image generates the contour line image with a different color for each gradation.

In accordance with the imaging method according to the thirty-first aspect, the boundary between each gradation in the gradation image becomes clear.

Thirty-Second Aspect In the imaging method according to any one of the twenty-sixth through the thirty-first aspects,

the predetermined region is an entire screen area of the fluorescent image.

In accordance with the imaging method according to the thirty-second aspect, it is possible to prevent, for example, oversight of the state of blood circulation depending on the case of the examinee.

Thirty-Third Aspect In the imaging method according to any one of the twenty-sixth through thirty-second aspects,

the predetermined region is a plurality of regions in the fluorescent image.

In accordance with the imaging method according to the thirty-third aspect, it is possible to improve, for example, the image processing speed.

Thirty-Fourth Aspect In the imaging method according to any one of the twenty-sixth through thirty-fourth aspects,

the imaging method further comprising capturing in real time a visible image in an area corresponding to the fluorescent image, wherein

the displaying a gradation image superimposes the gradation image on the visible image for display.

In accordance with the imaging method according to the thirty-fourth aspect, it is possible to readily visually recognize the boundary between each gradation in the gradation image.

1. An imaging device comprising:
an excitation light source configured to irradiate an examinee with excitation light to excite a fluorescent dye administered to the examinee;
a capturing unit configured to obtain a fluorescent image by capturing fluorescence generated from the fluorescent dye;
a pixel value measurement unit configured to measure, over time, a pixel value of each pixel in a predetermined region of the fluorescent image;
a gradation conversion unit configured to perform gradation conversion of the pixel values measured by the pixel value measurement unit to a predetermined number of gradations;
a gradation image generation unit configured to generate a gradation image based on the pixel values subjected to the gradation conversion by the gradation conversion unit; and
an image display unit configured to display the gradation image.

2. The imaging device according to claim 1, wherein the gradation image generation unit generates the gradation image with a different color for each gradation.

3. The imaging device according to claim 1, further comprising a contour line generation unit configured to detect a boundary between each gradation in the gradation image and generate a contour line image representing each gradation, wherein
the image display unit displays at least any one of the gradation image or the contour line image.

4. The imaging device according to claim 3, wherein the contour line generation unit generates the contour line image with a different thickness for each gradation.

5. The imaging device according to claim 3, wherein the contour line generation unit generates the contour line image with a different color for each gradation.

6. The imaging device according to claim 1, wherein the predetermined region is an entire screen area of the fluorescent image.

7. The imaging device according to claim 1, wherein the predetermined region is a plurality of regions in the fluorescent image.

8. The imaging device according to claim 1, wherein
the capturing unit captures a visible image in real time in an area corresponding to the fluorescent image, and
the image display unit superimposes the gradation image on the visible image for display.

9. An imaging device comprising:
an excitation light source configured to irradiate an examinee with excitation light to excite a fluorescent dye administered to the examinee;
a capturing unit configured to obtain a fluorescent image by capturing fluorescence generated from the fluorescent dye;
an image storage unit configured to store the fluorescent image over time;
a pixel value measurement unit configured to measure, over time, a pixel value of each pixel in a predetermined region of the fluorescent image based on the fluorescent image stored in the image storage unit;
a change curve creation unit configured to create a temporal change curve of the pixel value of each pixel in the predetermined region based on the pixel values measured by the pixel value measurement unit;
an index calculation unit configured to analyze the change curve of each pixel in the predetermined region and calculate a predetermined index for each pixel in the predetermined region;
a gradation conversion unit configured to perform gradation conversion of the predetermined index to a predetermined number of gradations;
a gradation image generation unit configured to generate a gradation image based on the index subjected to the gradation conversion by the gradation conversion unit; and
an image display unit configured to display the gradation image.

10. The imaging device according to claim 9, wherein the predetermined index is any one of gradient, a peak value, time to peak, an integral, or a curve shape of the change curve.

11. The imaging device according to claim 9, wherein the gradation image generation unit generates the gradation image with a different color for each gradation.

12. The imaging device according to claim 9, further comprising a contour line generation unit configured to detect a boundary between each gradation in the gradation image and generate a contour line image representing each gradation, wherein
the image display unit is capable of switching between the gradation image and the contour line image for display.

13. The imaging device according to claim 12, wherein the contour line generation unit generates the contour line image with a different thickness for each gradation.

14. The imaging device according to claim 12, wherein the contour line generation unit generates the contour line image with a different color for each gradation.

15. The imaging device according to claim 9, wherein the predetermined region is an entire screen area of the fluorescent image.

16. The imaging device according to claim 9, wherein the predetermined region is a plurality of regions in the fluorescent image.

17. The imaging device according to claim 9, wherein
the capturing unit captures a visible image in real time in an area corresponding to the fluorescent image, and
the image display unit superimposes the gradation image on the visible image for display.

18. An imaging method comprising the steps of:
irradiating an examinee with excitation light to excite a fluorescent dye administered to the examinee;
obtaining a fluorescent image by capturing fluorescence generated from the fluorescent dye;
measuring, over time, a pixel value of each pixel in a predetermined region of the fluorescent image;
performing gradation conversion of the measured pixel values to a predetermined number of gradations;
generating a gradation image based on the pixel values subjected to the gradation conversion; and
displaying the gradation image.

19. The imaging method according to claim 18, wherein the generating a gradation image generates the gradation image with a different color for each gradation.

20. The imaging method according to claim 18, further comprising detecting a boundary between each gradation in the gradation image and generating a contour line image representing each gradation, wherein
the displaying a gradation image displays at least any one of the gradation image or the contour line image.

21. The imaging method according to claim 20, wherein the generating a contour line image generates the contour line image with a different thickness for each gradation.

22. The imaging method according to claim 20, wherein the generating a contour line image generates the contour line image with a different color for each gradation.

23. The imaging method according to claim 18, wherein the predetermined region is an entire screen area of the fluorescent image.

24. The imaging method according to claim 18, wherein the predetermined region is a plurality of regions in the fluorescent image.

25. The imaging method according to claim 18, further comprising capturing a visible image in real time in an area corresponding to the fluorescent image, wherein
the displaying a gradation image superimposes the gradation image on the visible image for display.

26. An imaging method comprising the steps of:
irradiating an examinee with excitation light to excite a fluorescent dye administered to the examinee;
obtaining a fluorescent image by capturing fluorescence generated from the fluorescent dye;
storing the fluorescent image over time;
measuring, over time, a pixel value of each pixel in a predetermined region of the fluorescent image based on the fluorescent image subjected to the storing;
creating a temporal change curve of the pixel value of each pixel in the predetermined region based on the measured pixel values;
analyzing the change curve of each pixel in the predetermined region and calculating a predetermined index for each pixel in the predetermined region;
performing gradation conversion of the predetermined index to a predetermined number of gradations;
generating a gradation image based on the index subjected to the gradation conversion; and
displaying the gradation image.

27. The imaging method according to claim 26, wherein the predetermined index is any one of gradient, a peak value, time to peak, an integral, or a curve shape of the change curve.

28. The imaging method according to claim 26, wherein the generating a gradation image generates the gradation image with a different color for each gradation.

29. The imaging method according to claim 26, further comprising detecting a boundary between each gradation in the gradation image and generating a contour line image representing each gradation, wherein
the displaying a gradation image is capable of switching between the gradation image and the contour line image for display.

30. The imaging method according to claim 29, wherein the generating a contour line image generates the contour line image with a different thickness for each gradation.

31. The imaging method according to claim 29, wherein the generating a contour line image generates the contour line image with a different color for each gradation.

32. The imaging method according to claim 26, wherein the predetermined region is an entire screen area of the fluorescent image.

33. The imaging method according to claim 26, wherein the predetermined region is a plurality of regions in the fluorescent image.

34. The imaging method according to claim 26, further comprising capturing in real time a visible image in an area corresponding to the fluorescent image, wherein
the displaying a gradation image superimposes the gradation image on the visible image for display.
